(19) United States
(12) Patent Application Publication
(10) Pub. No.: US 2009/0056213 A1
(43) Pub. Date: Mar. 5, 2009

(54) Method for repairing, regrassing or establishing a green

(76) Inventors: Wendy Van Paridon-Bakker, Wijdenes (NL); Stuart William Akers, Gilroy, CA (US)

Correspondence Address: TRASK BRITT, P.O. BOX 2550, SALT LAKE CITY, UT, 84110, US

(21) Appl. No.: 11/920,027

(22) PCT Filed: May 4, 2006
(86) PCT No.: PCT/NL2006/000238

§ 371 Date: May 22, 2008

(51) Int. Cl. A01C 1/00; A01H 5/10

(52) U.S. Cl. 47/58.1 R; 800/298; 47/58.1 SE

(57) ABSTRACT

The invention provides a method for repairing, regrassing or establishing a green, which method comprises incorporating in the soil surface of the green and/or putting in holes in the surface of the green an/or applying at the soil surface of the green a flowable primed grass seed which displays physiological activity. The invention also relates to the use of primed grass seed to repair, regrass or establish a green which grass seed displays physiological activity. Further, the invention provides a flowable primed grass seed displaying physiological activity.

The present invention relates to a method for rapidly repairing, regrassing or establishing a green, the use of a particular grass seed to rapidly repair, regrass or establish a green, and a flowable primed grass seed which displaying physiological activity.

In the sports and leisure industry there is a need for well-maintained high quality greens, such as golf greens, cricket green including pitches, bowling greens, tennis courts, home lawns, soccer fields, parks and so on. Especially in sports, the greens can be damaged relatively easy, which often make them unsuitable for continued use. Hence, there is clear demand for equipment and processes to repair greens very quickly, allowing the green in question to be used again without any unduly delay.

It is known to repair damaged greens or greens that otherwise are in need of repair with grass seed that has been primed. The term “priming” is well understood and defines a pre-sowing hydration treatment of seeds, which is carried out in the presence of restricted amounts of water with or without additional chemicals. Priming is designed to increase germination speed, germination uniformity and also to break all kinds of dormancies. Seed does not sprout during priming, but when priming is completed the primed seed is very close to that end point of germination. Conventionally, once the priming process is completed, the seed is dried to the appropriate low moisture content that facilitates a sufficiently long shelf life. However, a considerable drawback of conventional primed and dried grass seeds is that they do not allow for as rapid repair since such primed and dried seed must re-imbibe water to re-initiate physiological activity within the seed which process takes a relatively long period of time. Therefore, conventionally primed and dried grass seed does not allow a rapid repair of a green.

Object of the present invention is to provide a method, which enables a very rapid repair of a green, especially so-called high value greens such as golf greens, and in particular the areas of driving ranges where golf shots are practised.

Surprisingly, it has now been found that this can be realised when use is made of a primed grass seed, which is flowable and displays physiological activity.

Accordingly, the present invention relates to a method for repairing, regrassing or establishing a green, which method comprises incorporating in the soil surface of the green and/or putting in holes in the soil surface of the green and/or applying at the soil surface of the green a flowable primed grass seed which displays physiological activity.

The primed grass seed to be used in accordance with the present invention can be incorporated in the soil of the surface of the green to be repaired, regrassed or established. It can be applied at the soil surface of the green, or it can be put in holes in the soil surface of the green. Preferably, the primed grass seed is incorporated in the soil surface of the green to be repaired, regrassed or established. Such incorporation of the grass seed into greens can suitably be established by means of any known planting method. Preferably, such incorporation is established by means of a drop seed planter after which a rake can be applied to mix the grass seed with loose soil on the surface of the green to be repaired. Subsequently, a light cover of an appropriate material such as redwood mulch can be applied, after which the green can be watered.

Holes can be prepared in the soil surface of the green by means of an apparatus that punches holes in the soil surface of the green.

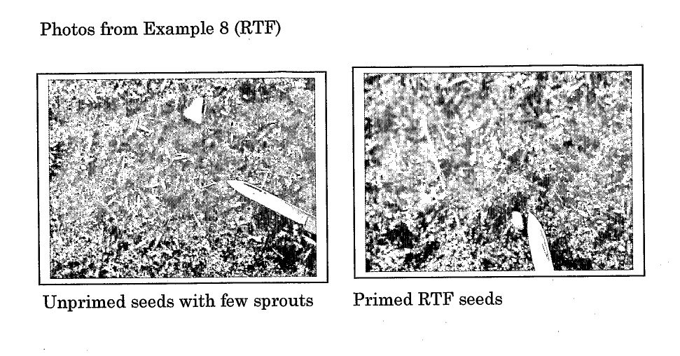

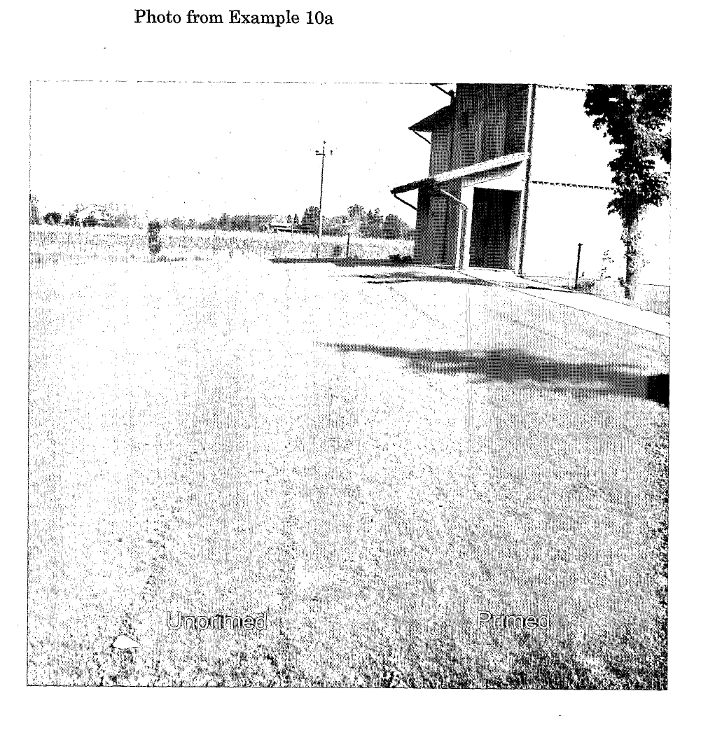

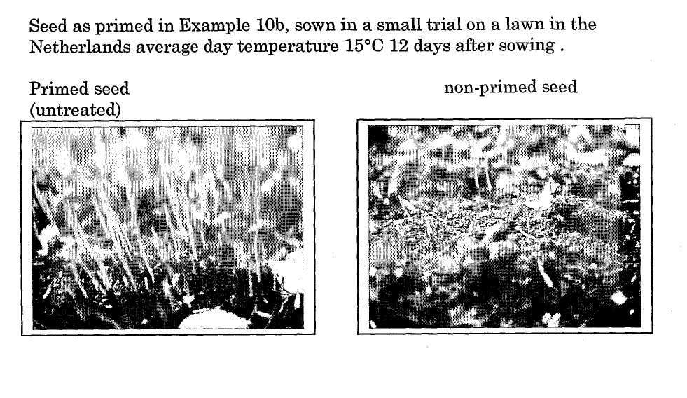

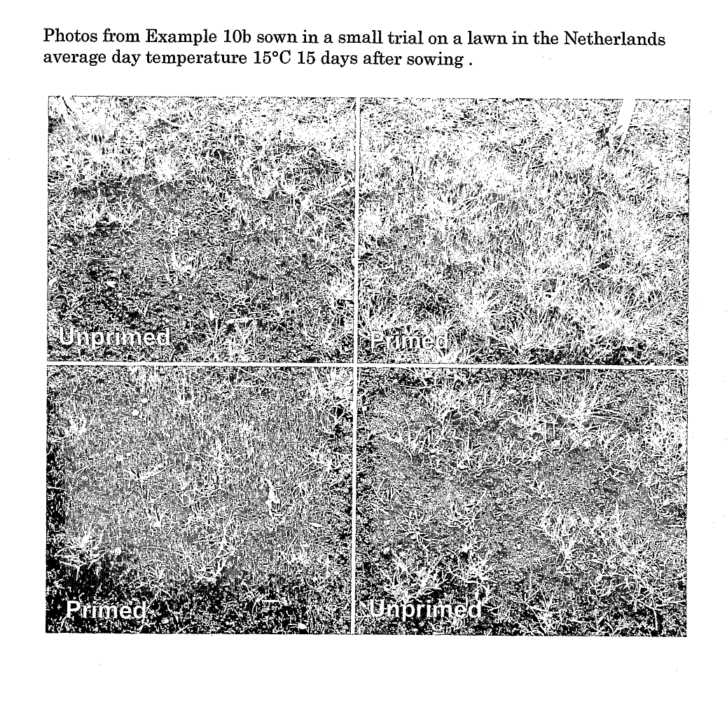

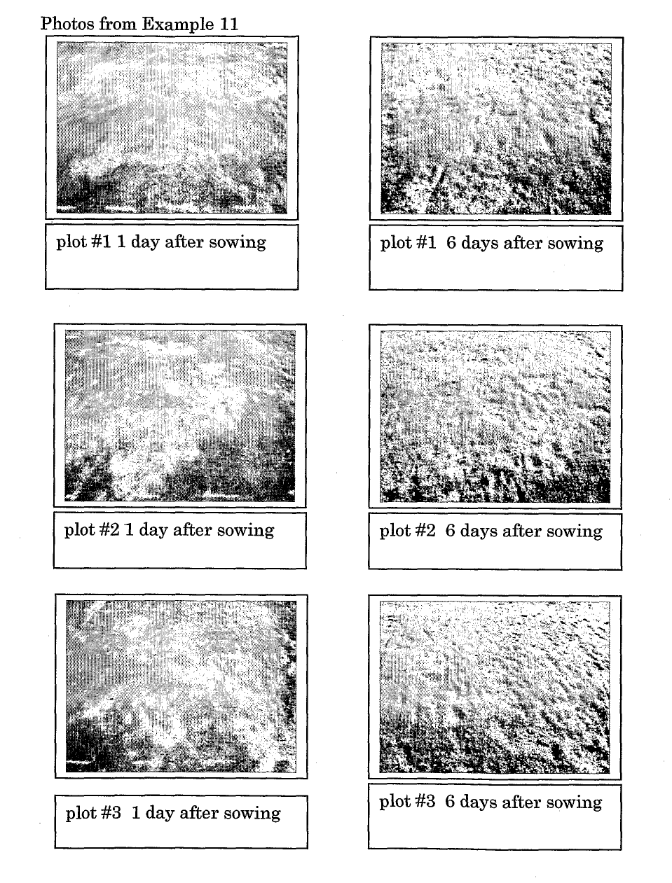

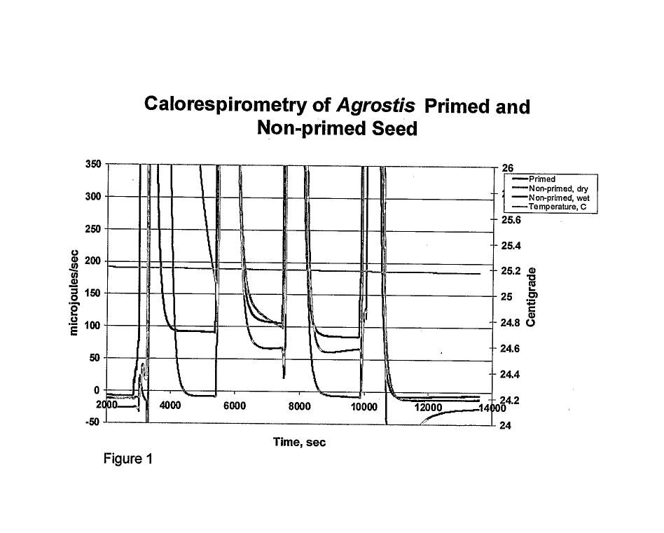

In order to avoid any misunderstanding it is noted that the flowable primed grass seed displays physiological activity before it is incorporated in the soil surface of the green and/or put in holes in the soil surface of the green and/or applied at the soil surface of the green. In other words, the physiological activity developed within the seed during the priming process was not lost since the seed was not dried back to a low moisture content. In this respect it is observed that physiological activity relates to the activity of fundamental processes such as photosynthesis, respiration and transpiration. As such it is well known that physiological activity in seeds is affected by water content (see C. W. Vertucci and A. C. Leopold, Plant Physiol. (1987) 84, 1038-1043). It is understood that physiological activity can be determined by means of calorimetric measurements. See, for example, FIG. 1.

The primed grass seed to be used in accordance with the present invention is flowable, i.e. it is substantially surface dry. In other words, the seed particles can freely move with respect to each other. This can suitably be established by partial dehydration of the freshly primed seed or by applying a coating on the freshly primed seed. Preferably, the entire surface of the individual seeds is dry. The use of a flowable primed grass seed that displays physiological activity enables a surprisingly rapid repair of a green.

The flowable primed grass to be used in accordance with the present invention is obtained by subjecting a grass seed to a priming process. In case the freshly primed seed so obtained is flowable, it can advantageously immediately be used to repair, regrass or establish a green. Alternatively, the freshly primed grass seed may need to be dried partially before it can be used repair, regrass or establish a green. The flowable primed grass seed to be used in accordance with the present can also suitably be stored at low temperature for a certain amount of time before it is use to repair, regrass or establish a green. The flowable primed grass seed to be used in accordance with the present invention will have a relatively high water content, but generally no germination will have yet occurred, i.e. the seed will usually display very few, if any radicles. Suitably, less than 1% of the flowable primed grass seed displays radicles. Preferably, less than 0.5% of the flowable primed grass seed displays radicles. In a particular attractive embodiment the flowable primed grass seed displays in essence no radicles. This unique combination of high water content and absence of radicles allows for the surprisingly rapid repair, regrass treatment or establishment of a green, which is especially advantageous in respect of golf greens that need to be used almost continuously, in particular the areas of a teeing ground, fairway and putting green. Preferably, the flowable primed grass seed does not display any radicles. It is observed, however, that improved results are also obtained with flowable primed grass seed according to the invention that display quite a number of radicles, i.e. up to 50% radicles.

The grass seed to be used in accordance with the present invention displays improved earliness, as well as an improved uniformity of final stand at both low (below 20° C.) and high temperatures (above 20° C.).

Suitably, the grass seed to be primed has a water content in the range of from 5 to 15% wt, based on total weight of seed. Preferably, the primed grass seed has a water content in the range of from 25 to 50% wt, based on total weight of seed. The skilled person will understand that the water content to be obtained will depend on the amount of water to be used in the priming process and/or the length of time during which the grass seed is subjected to the priming process and/or the grass species which is primed and or the used chemicals. Hence, the skilled person will know how to obtain primed seeds having a particular water content.

A variety of known priming processes can be used in accordance with the present invention. Such priming processes include the use of osmopriming, solid matrix priming, drum priming or any other priming method.

Suitably, the primed grass seed has such a water content that the ratio of the water contents of the primed grass seed (A) and the raw untreated grass seed to be primed (B) is greater than 1 (A/B). Preferably, A/B is in the range of from 1.6 to 10, more preferably in the range of from 2 to 5, more preferably in the range of from 2.5 to 4. Such grass seeds allow for a very attractive and swift repair of greens such as golf greens, cricket green including pitches, bowling greens, tennis courts, home lawns, and parks, which is especially of importance for greens that are very frequently used such as golf greens. Hence, the method is preferably carried out to repair a golf green.

After the primed grass seed to be used in accordance with the present invention has been prepared, it can very quickly be used to repair, regrass or establish a green. Preferably, the grass seed is used within 100 hours after the priming process has been completed. More preferably, the primed grass seed is used within 24 hours after the priming process has been completed. This period will depend on the temperature of storage of the primed grass seed, as well as its water content. At low temperature (5° C.) the time to use the primed seeds can be elongated. The time for use to repair the green can be increased by shortening the priming period.

In accordance with the present invention a wide variety of grass seeds within the plant order of Poales specifically within the familie Poaceae can be used. This family contain over 12000 species. Suitable types of grass seeds include those selected from the group consisting of Poa, Lolium, Dactylis, Festuca, Deschampsia, Koeleria, Agrostis, Cynodon, Zoysia, Buchlo, Axonopus, Eremchloa, Paspalum and Stentaphrum.

Very attractive results are obtained when the grass seed comprises Poa pratensis, Lolium perrenne, Agrostis capillaris tenuis, Festuca rubra or Festuca arundinacea.

Also mixtures of two or more types of grass seed can be used in accordance with the present invention. Such seed mixtures are especially suitable for soccer fields.

The primed grass seed may be treated with any known additives that are usually used to improve the quality of the green. Suitable additives include fungicides, insecticides, bactericides, nematicides, biocides and herbicides (weed killers). In addition, growth promotive additives such micro and/or macro nutritients, fertilizers, hormones, bioenhancers, gen-elicitors, plant strengtheners, plant or seaweed extracts, such as neem extracts, peroxides or even benificial micro organisms such as Bacillus, Trichoderma or Pseudomonas can be used. Such additives can suitably be present in an amount in the range of from 0.001 to 100% wt, based on total weight of seed. Suitably, use can be made of selective fertilizers. Such selective fertilizers promote selectively the growth of one or more particular types of grass when compared to other types of grass. Such additives can be part of a coating applied on the seed particles. Such additives can also be applied separatedly or together with the seed. Accordingly, the present invention also relates to a method according to the invention wherein in addition an additive to improve the quality of the grass is incorporated in the soil surface of the green and/or put in holes in the soil surface of the green and/or applied at the soil surface of the green.

In addition, particles (e.g. granulates) of a super absorbent polymer (a superslurper) can be applied in a similar manner. Hence, the present invention also relates to the method according to the present invention wherein in addition particles of a super absorbent polymer (a superslurper) are incorporated in the soil surface of the green and/or put in holes in the soil surface of the green and/or applied at the soil surface of the green. The particles of the super absorbent polymer (a superslurper) act as a moisture source for the primed grass seed.

Suitably, the primed grass can be coated before it is used to repair a green. The coating can be established by means of any of the known coating processes that are used for that purpose. This coating layer can improve the flowability and therefore the plantability of the primed seeds. Suitable coating methods include film coating, encrusting or pelleting. Preferably, the coating of the primed grass seed is established by an encrusting method. Such a coating can suitably be present in an amount in the range of from 0.1 to 1000% wt, based on total weight of seed.

Suitably, the primed grass can be premixed with particulate materials to enhance the flowability in order to improve the plantability. Suitable materials, but not limited to these examples, include talcum, fumed silica, mica, vermiculite, perlite and/or sand.

In a particular attractive embodiment of the present invention, the priming process is carried out in a transferable unit (e.g. a truck), enabling the grass seed to be used for repairing, regrassing or establishing a green within just a few hours after the priming process has been completed.

The present invention also relates to the use of primed grass seed for repairing, regrassing or establishing a green. The grass seed may be any of the type of grass seeds described above, and these seeds can be primed using the type of processes indicated before.

The present invention also relates to a flowable primed displaying physiological activity. Preferably, the flowable primed grass seed displays physiological activity to the extent that it is completely ready for germination, but very little, if any, radicle protrusion has taken place. Preferably, said primed grass seed has a water content in the range of from 25 to 50% wt, based on total weight of seed.

The flowable primed grass seed in accordance with the present invention can be used to repair, regrass or establish a green.

Hence, the present invention provides a method for repairing a green, which method comprises incorporating in the soil surface of the green and/or putting in holes in the soil surface of the green and/or applying at the soil surface of the green to be repaired a flowable primed grass seed, which displays physiological activity.

In addition, the present invention also provides a method for regrassing a green, which method comprises incorporating in the soil surface of the green and/or putting in holes in the soil surface of the green and/or applying at the soil surface of the green to be repaired a flowable primed grass seed, which displays physiological activity. The flowable primed grass seed in accordance with the present invention can be used to regrass an existing green completely in case the turf has been removed from the soil. A particular advantage of the flowable primed grass seed of the present invention that it allows over time the complete replacement on a green of one type of grass by another type of grass, which obviously saves a lot of time (and money), which is especially of importance of frequently used greens such as teeing grounds, fairways and putting greens. In other words, in the context of the present invention the term regrassing also covers the replacement of one or more types of grass by one or more other types of grass.

Further, the present invention also provides a method for establishing a green, which method comprises incorporating in the soil surface of the green and/or putting in holes in the surface of the green and/or applying at the soil surface of the green to be repaired a flowable primed grass seed, which displays physiological activity. In other words, the present invention allows for the establishment of a green starting just from bare soil. Also, this embodiment is very attractive since a very rapid building of a green can be realised.

### EXAMPLES

### Example 1

A method according to the present invention was carried out as follows. 10 g of Bent grass seed (Agrostis capillaris) having a water content of 9.48% wt, based on total weight of seed were subjected to the following priming process. In a 250 ml container, 10 g of solid matrix material AGROLIG (Natural Crude Humic Acid, Americal colloid company Illenois) was blended with 6 g of softened water containing 0.2% KNO₃, and thoroughly stirred with a spatula. Then the 10 g of Bentgrass seed was added, and again thoroughly stirred with a spatula. The container was closed with a lid in which an aeration hole of about 1.5 mm diameter was made. The container was located in a 20° C. climate chamber with continuously fluorescent light on a roller belt providing rotation movement to the container. After 4 days the priming was terminated. A total of 22.0 g of the total weight of 25.4 g of the mixture so obtained (seed/matrix/sol) was sieved above a mesh screen to separate the seed from the matrix. A weight of 14.8 g of non-dried primed seed with some attached matrix was obtained. The seeds were directly planted in a germination test. In Table 1, experimental data are shown of the non-dried primed seed in accordance with the invention, together with data on untreated seed. From these data it will be clear that that the primed grass seed according to the invention displays a significant improved over the untreated seed in terms of germination speed (see the respective sprout counts).

TABLE 1

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 0 | 24 | 58 | 55 | 79 | 88 | 76 | 76 | 36 | 0 |
| untreated seed | 0 | 0 | 0 | 0 | 9 | 42 | 39 | 15 | 0 | 0 |

Thermogradient test - Sprout counts made after 2 days. Thermogradient range is from 16° C. to 36° C.

### Example 2

A method according to the present invention was carried out as follows. 4.1 kg of a Perennial rye grass blend (Lolium perenne) having a water content of 10.5% wt, based on total weight of seed was subjected to a priming process similar to Example 1 in that at the end of priming the primed seed had a high moisture content, was still flowable and had less than 1% sprouted seed. The seed was primed for 4.75 days. For comparison purposes, some of the seed was dried to its original (pre-priming) moisture content. After the priming process was completed the primed grass blend was immediately recovered and stored in a container kept at 15° C. Four hours later the grass seed so obtained was planted in a green in need of repair. The seed was planted at a rate of ½ lb per plot (plots were 3 feet by 6 feet=18 sq feet). Each plot was planted within 15 minutes using a seed planter. Then a rake was used to mix the seed with loose soil on the surface and a light cover of redwood mulch was applied, after which the plot was thoroughly watered. For reasons of comparison, grass seed that had been primed and dried in a conventional manner was also planted, as well as seed that had not been primed (untreated seed).

After 3 days green grass sprouts came up through the redwood mulch in the plot where freshly primed seed in accordance with the present invention had been planted. It was 2 days later before sprouts were seen in the plot with the conventionally primed and dried seed, as well as the plot with the non-primed seed.

The germination of each of these three seeds was also conducted, using a thermogradient table. In this test, a temperature gradient was formed on a table such that 10 plantings could be made into 10 different temperatures (16-36° C.). Once planted, the seeds were germinated in the dark (see Tables 2 and 3 below).

From the results, shown in Tables 2 and 3, it will be clear that the method according to the present invention constitutes a significant improvement over the method wherein use is made of a primed grass which has subsequently be subjected to a drying step, resulting in a grass seed having a low water content.

TABLE 2

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 67 | 72 | 79 | 82 | 82 | 85 | 82 | 76 | 70 | 18 |
| Primed & dried | 6 | 24 | 36 | 36 | 64 | 61 | 70 | 64 | 42 | 0 |
| untreated seed | 0 | 3 | 12 | 39 | 42 | 42 | 42 | 46 | 12 | 0 |

Thermogradient test - Sprout counts made after 2 days. Thermogradient range is from 16° C. to 36° C.

TABLE 3

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 85 | 85 | 88 | 76 | 85 | 88 | 88 | 33 | 24 | 0 |
| Primed & dried | 12 | 55 | 76 | 70 | 82 | 79 | 94 | 76 | 15 | 0 |
| untreated seed | 9 | 70 | 91 | 91 | 94 | 85 | 88 | 58 | 0 | 0 |

Thermogradient test - Counts of seedlings with a visible coleoptile were made after 5 days on the table. TG range is from 16° C. to 36° C.

### Example 3

A method according to the present invention was carried out as follows. 92 kg of Bent Grass (Seaside II) having a water content of 10.96% wt, based on total weight of seed, was subjected to a priming process similar to Example 1 in that at the end of priming the primed seed had a high moisture content, was still flowable and had less than 1% sprouted seed. The seed was primed for 7 days. After the priming process was completed the primed grass seed was immediately recovered and stored in a container kept at 15° C. Two hours later the freshly primed grass seed was planted on a green.

The purpose of this planting was to replace the Poa grass (Poa annua ???? annual and perennial) that is currently present on the green with Bent grass. The primed bent grass seed was planted into the green that is currently populated with Poa. Within a year after first sowing a substantial part (at least 25%) of the Poa grass was replaced by bent grass.

### Example 4

A method according to the present invention was carried out as follows. 11.34 kg of Rough blue grass seed (Poa trivialis) was primed according to the method similar to Example 1 in that at the end of priming the primed seed had a high moisture content, was still flowable and had less than 1% sprouted seed After 7 days of priming, the germination results were obtained as shown in Table 4, together with the results obtained with untreated seed. From these data it will be clear that under sub-optimal conditions improved results are obtained with seed according to the present invention.

TABLE 4

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 15 | 33 | 46 | 67 | 52 | 76 | 70 | 52 | 34 | 0 |
| untreated seed | 6 | 27 | 61 | 52 | 73 | 61 | 55 | 30 | 6 | 0 |

Thermogradient test - Sprout counts made after 2 days. Thermogradient range is from 16° C. to 36° C.

### Example 5

A method according to the present invention was carried out as follows. 50 g of Kentucky Blue Grass seed (Poa pratensis) was primed according to the method similar to Example 1 in that at the end of priming the primed seed had a high moisture content, was still flowable and had less than 1% sprouted seed. After 7 days of priming, the germination results were obtained as shown in Table 5, together with the results obtained with untreated seed. From these data it will be clear that with the seed according to the present invention significantly improved results are obtained when compared with untreated seed.

TABLE 5

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 61 | 73 | 70 | 52 | 76 | 52 | 82 | 27 | 9 | 0 |
| untreated seed | 3 | 9 | 18 | 18 | 9 | 3 | 0 | 0 | 0 | 0 |

Thermogradient test - Sprout counts made after 2 days. Thermogradient range is from 16° C. to 36° C.

### Example 6

A method according to the present invention was carried out as follows. 80 kg of creeping bent grass (Penn G-6) seed was primed according to the method similar to Example 1 in that at the end of priming the primed seed had a high moisture content, was still flowable and had less than 1% sprouted seed. After 6 days of priming, the germination results were obtained as shown in Tables 6-8.

TABLE 6

| | Temp. (° C.) | | | | | | | | | | |
|---|---|---|---|---|---|---|---|---|---|---|---|
| Treatments | 16-18 | 18-20 | 20-22 | 22-24 | 24-26 | 26-28 | 28-30 | 30-32 | 32-34 | 34-36 | Average |
| Primed not | 24 | 46 | 67 | 61 | 70 | 61 | 64 | 21 | 18 | 3 | 44 |
| dried | | | | | | | | | | | |
| Untreated | 0 | 21 | 21 | 42 | 27 | 9 | 12 | 15 | 6 | 0 | 15 |
| seed | | | | | | | | | | | |

Thermogradient test - Sprout counts made after 3 days. Thermogradient range is from 16° C. to 36° C.

TABLE 7

| | Temp. (° C.) | | | | | | | | | | |
|---|---|---|---|---|---|---|---|---|---|---|---|
| Treatments | 16-18 | 18-20 | 20-22 | 22-24 | 24-26 | 26-28 | 28-30 | 30-32 | 32-34 | 34-36 | Average |
| Primed not | 58 | 61 | 61 | 61 | 70 | 61 | 58 | 24 | 27 | 3 | 48 |
| dried | | | | | | | | | | | |
| Untreated | 27 | 45 | 27 | 52 | 27 | 9 | 12 | 18 | 6 | 0 | 22 |
| seed | | | | | | | | | | | |

Thermogradient test - Counts of seedlings with a visible coleoptile made after 7 days. Thermogradient range is from 16° C. to 36° C.

TABLE 8
Thermogradient table test -tested after 15 days storage at 5° C. -
counted 7 days after planting.

| | Temp. | | | | | | | | | | |
|---|---|---|---|---|---|---|---|---|---|---|---|
| Treatments | 16-18 | 18-20 | 20-22 | 22-24 | 24-26 | 26-28 | 28-30 | 30-32 | 32-34 | 34-36 | Ave |
| Primed not | 42 | 52 | 61 | 76 | 73 | 70 | 76 | 79 | 42 | 15 | 59 |
| dried | | | | | | | | | | | |
| Untreated | 27 | 21 | 49 | 36 | 42 | 21 | 36 | 18 | 6 | 0 | 26 |
| seed | | | | | | | | | | | |

Table 8 shows that the priming process enhanced the performance of the seed by improving temperature tolerance and by increasing the speed of germination as well as final germination. In addition, storage at 5° C. appeared to improve the seed tolerance to high temperature, but the seed also seemed to loose some of the advantage it had gained at low temperatures.

From Tables 6-8 it will be clear that the primed seed according to the invention displayed an improved germination speed. In addition, the sub-optimal temperatures (32/34° C. and 16/18° C.) show improved emergences.

### Example 7

A method according to the present invention was carried out as follows. 695 g of creeping bent grass (Penn A-4) seed was primed according to the method as previously described in Example 1. After 6 days of priming, the germination results were obtained as shown in Tables 10 and 11.

TABLE 9
Thermogradient table test - Planted 4 days after priming was
terminated (held at 5 C. until planted). - counted at day 3 after planting

| | Temp. (° C.) | | | | | | | | | | |
|---|---|---|---|---|---|---|---|---|---|---|---|
| Treatment | 16-18 | 18-20 | 20-22 | 22-24 | 24-26 | 26-28 | 28-30 | 30-32 | 32-34 | 34-36 | Ave |
| Primed not | 33 | 39 | 58 | 42 | 61 | 61 | 70 | 58 | 39 | 9 | 47 |
| dried | | | | | | | | | | | |
| Untreated | 0 | 30 | 21 | 36 | 27 | 30 | 15 | 6 | 3 | 0 | 17 |
| seed | | | | | | | | | | | |

TABLE 10
Thermogradient table test - Planted 4 days after priming was
terminated (held at 5 C. until planted). Counted day 7 after planting.

| | Temp. (° C.) | | | | | | | | | | |
|---|---|---|---|---|---|---|---|---|---|---|---|
| | 16-18 | 18-20 | 20-22 | 22-24 | 24-26 | 26-28 | 28-30 | 30-32 | 32-34 | 34-36 | Ave |
| Primed not | 64 | 55 | 76 | 61 | 61 | 61 | 70 | 64 | 39 | 15 | 57 |
| dried | | | | | | | | | | | |
| Untreated | 52 | 58 | 52 | 46 | 36 | 33 | 18 | 9 | 6 | 0 | 31 |
| seed | | | | | | | | | | | |

The obtained primed seeds were planted together with non-primed seeds in a field trial on a golf course located in California (US) in the following manner.

Two sowing exercises were carried out:

1. Non-primed (untreated) seed was planted at an amount of 1 lb per 1000 ft².
2. Primed—not dried—seed (having a weight increase due to the priming process) was planted at an amount of 1.12 lb per 1000 ft2.

The following two cover layers were used:
1. sand; and
2. sand and superabsorbent ZEBA.

Plots sizes were 3 ft². Non-primed (untreated) seed was used in an amount of 1.36 g, whereas primed seed was used in an amount of 1.53 g. Three replicates were carried out per treatment. The blocks were assessed under application of code-numbers to ensure that proper and objective assessments could be made.

The results as shown in Table 12 were obtained from visual assessment:

TABLE 11
Visual estimates as % coverage made for three dates:

| | March | March | March |
|---|---|---|---|
| Treatment | 3rd | 21st | 30th |
| C + S | 8 | 40 | 55 |
| P + S | 8 | 47 | 85 |
| C + Z | 35 | 67 | 88 |
| P + Z | 37 | 70 | 92 |

C = nonprimed seed
P = primed-non dried seed
S = sand coverage layer
Z = sand + ZEBA coverage layer

The test results in Table 11 indicate that priming of the seed increases the speed of germination and the temperature tolerance of the seed (relative to germination). The visual estimates indicate that the field planting obviously provided stresses other than temperature. Since the addition of ZEBA to the planting dramatically improved the early emergence, it would appear that the stress that was dominant in this trial was associated with dehydration.

In co-operation with the University of Arkansas a digital analysis method of the turf grass coverage has been developed, which analysis method is used in the Example. For details of this analysis method reference is made to M. D. Richardson, D. E. Karcher and L. C. Purcell, Crop Sci. 41:1884-1888 (2001)). The results are shown in Table 12 confirming the earlier findings from the visual assessment.

TABLE 12
Quantified % coverage based on digital analysis of the green surface
area for 7 dates

| | Febr | Febr | Febr | Mar | | | |
|---|---|---|---|---|---|---|---|
| Treatment | 17th | 21st | 27th | 3th | Mar 8th | Mar 21st | Mar 30th |
| C + S | 1 | 4 | 5 | 8 | 15 | 25 | 53 |
| P + S | 1 | 5 | 6 | 9 | 17 | 31 | 68 |
| C + Z | 1 | 6 | 9 | 19 | 29 | 54 | 79 |
| P + Z | 2 | 7 | 12 | 21 | 26 | 51 | 75 |

### Example 8

A method according to the present invention was carried out as follows. 11 kg of Rhizomatous tall fescue (RTF) seed with a water content of 9.74% wt was primed according to a method similar to Example 1 in that at the end of priming the primed seed had a high moisture content, was still flowable and had less than 1% sprouted seed. After 7 days of priming, the seed was planted in the field, i.e. a driving range of a golf course located in California (USA).

The following results of visual assessment were obtained after 11 days. Photos of the results are attached.

| | % of emergence |
|---|---|
| Primed non-dried RTF | 60 |
| Non primed RTF | 5 |

From these results it will be clear that the primed grass seed in accordance with the invention displayed a considerable improved emergence.

### Example 9

Seeds as obtained in Example 1 were planted in sand under the following laboratory conditions. Germination was executed in sand having a specification size of 0.2-0.63 mm and having a water content of 0.12% wt. The following amounts of water were respectively added to the sand: 10%, 12.5% and 15% of the maximum water holding capacity of the sand which was 22% wt.

Three germination boxes were separated in 6 equal compartments with a surface area of 184 cm² each. For each box 6000 g of dry sand was prepared to which respectively 10%, 12.5%, and 15% of water was added (i.e. respectively. 132 g, 165 g, 198 g of water). These sand mixtures thus obtained were each put in the germination boxes (three in total) and divided among the 6 compartments, whereby each germination box contained 3 compartments with original (untreated) seed, and 3 compartments with primed seed according to the invention.

In each compartment on average 156 seeds were spread onto the sand surface. In each box, a first compartment with primed seed and a second compartment with untreated seed were covered with 10 g of sand. A third compartment with primed seed and a fourth compartment with untreated seed were covered with 10 g and 0.15 g of AquaZorb (a superslurper from Absorbent Technologies, Inc. Beaverton, Oreg. 97008), and a fifth compartment with primed seed and a sixth compartment with untreated seed were covered with 8 g of sand, 2 g of Chabamin J and 0.15 g AquaZorb.

Chabamin J (from société des Minerais de la Méditerranée SA, France) is a zeolite mainly made of a crystalline honeycomb structure (aluminium silicate) from alterated volcanic rock. This compound was added to improve the water absorption capacity of the surface layer.

TABLE 13
Percentage emergence determined 7 days after sowing in
15/25° C. 8 h light/16 h dark.

| | | % emergence after 7 days | | |
|---|---|---|---|---|
| | | | cover layer | |
| | % WHC of | | 2 sand + | 3 sand + AquaZorb + |
| Object | max. | 1 sand | AquaZorb | ChabaminJ |
| orig seed | 10.0 | 4 | 0 | 0 |
| orig seed | 12.5 | 0 | 0 | 0 |
| orig seed | 15.0 | 12 | 0 | 24 |
| primed | 10.0 | 3 | 0 | 2 |
| seed | | | | |
| primed | 12.5 | 21 | 1 | 4 |
| seed | | | | |
| primed | 15.0 | 92 | 21 | 99 |
| seed | | | | |

The results as shown in Table 13 indicate that the cover layer in which Aquazorb alone was included, resulted in a decreased emergence when compared with cover layer containing only sand. Aquazorb together with the sand developed a firm layer through which no radicle could protrude. Addition of the Chabamin J together with the Aquazorb to the sand resulted in two cases (original seed 15% and primed seed 15%) in an increased emergence compared to sand only.

From the data in Table 13 it will also be clear that primed seed performed better than the non primed seed at relatively high water content. The benefit of priming was especially visible at a WHC (water-holding content) of 12.5% of the maximum WHC of sand, which seemed to be a critical level. Non primed seed failed to germinate at this amount of water.

### Example 10a

A mixture of various grass seed, with a composition of 35% Perennial Rye (Lolium perenne), 20% Kentucky Blue Grass (Poa pratensis) and 45% Red Fescue (Festuca rubra) was separated into its respective grass seed components with mechanical upgrading devices (calibration on slot screen, and separation of one fraction with an air-separator). We obtained a fraction having an average particle size of more than 0.8 mm (21%; mainly Perennial Rye Grass); a fraction having an average particle size of between 0.60 and 0.80 mm (61%; mainly red fescue); and a fraction having an average particle size of less than 0.6 mm (18%; mainly Kentucky blue grass).

Since the various grass seed components show different speeds of emergence, for optimal results, the respective components of the seed mixture need to be primed during over different periods of time.

Red fescue was primed for 3 days; the Kentucky blue grass was primed during 7 days; and the perennial rye grass was only primed for one day. After mixing of the respective grass seed components back to the ratios of the original seed mixture, the seed mixture obtained was packed in polypropylene bags and put in cold storage boxes that contained ice packs. At 4 days after termination of the priming process, the primed seed was sown on a bare field of soil, and for reasons of comparison, another bare field of soil was sown with untreated seed.

Tables 14-16 show again that with the primed seed displays an improved germination speed.

### Red Fescue:

TABLE 14

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 30 | 30 | 33 | 33 | 36 | 42 | 36 | 21 | 12 | 0 |
| untreated seed | 0 | 3 | 12 | 9 | 12 | 18 | 15 | 6 | 3 | 0 |

Thermogradient test - Sprout counts made after 2 days. Thermogradient range is from 16° C. to 36° C.

### Kentucky Blue Grass

TABLE 15

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 12 | 15 | 15 | 24 | 21 | 21 | 42 | 18 | 9 | 0 |
| untreated seed | 6 | 12 | 12 | 24 | 24 | 12 | 12 | 0 | 0 | 0 |

Thermogradient test - Sprout counts made after 5 days. Thermogradient range is from 16° C. to 36° C.

### Perennial Rye Grass

TABLE 16

| | Temp. range ° C. | | | | | | | | | |
|---|---|---|---|---|---|---|---|---|---|---|
| Treatments | 16-18 | 18-20 | 20-22 | 22-24 | 24-26 | 26-28 | 28-30 | 30-32 | 32-34 | 34-36 |
| Primed not dried | 9 | 12 | 33 | 12 | 27 | 30 | 33 | 24 | 12 | 0 |
| untreated seed | 0 | 0 | 3 | 24 | 36 | 36 | 30 | 18 | 0 | 0 |

Thermogradient test - Sprout counts made after 2 days. Thermogradient range is from 16° C. to 36° C.

Field planting was done in Italy, 30 km away from Mirandola.

The amount of seed sown per m² was for non-primed seed about 28 g, and for primed seed 36 g. The sowing was executed so as to obtain with plots of 1.80 m wide per object.

The seed was then covered with soil by means of the sowing machine itself. Directly after sowing the field was irrigated, and this was repeated two days later. The average temperature during day time was about 30° C., in and 12° C. in the night.

Three days after sowing, the plot with primed seed showed a considerable number of sprouts, whereas in the plot with the untreated seed sprouts only started to appear six days after sowing (see also photo taken after 12 days).

### Example 10b

Both unprimed and primed seeds as described and prepared under Example 10a were sown in an poorly established green in The Netherlands, Enkhuizen. The amount of seed sown per m² was for non-primed seed about 35 g, and for primed seed 30 g. The sowing was executed so as to obtain small plots of 1 m² with 2×0.25 m² per object unprimed and primed (see photos).

The seeds were raked into the subplots and covered with potting soil and irrigated once directly after field bed preparation. The average temperature during day time was about 14° C. and 8° C. in the night.

At 7 days after sowing, the subplots with primed seed showed an emergence of about 40% sprouts of the finally observed number, whereas in the subplots with the untreated no seed sprouts were observed yet.

At 12 days after sowing, the subplots with primed seeds showed an emergence of about 95% sprouts of the finally observed number, whereas in the subplots with the untreated seed sprouts has just started to emerge and showed an emergence of about 25% of the finally observed number. (see photos)

At 14 days after sowing the subplots with the primed seeds showed clearly established grass seedlings filling up the bare spots where the overall impression of the subplots with unprimed seed was still rather bare and poorly established (see photos).

### Example 11

A method according to the present invention was carried out as follows. 10.8 kg of Perennial ryegrass blend seed having a water content of 11.32% wt, based on total weight of seed, was subjected to a priming process using a method similar to Example 1 in that at the end of priming the primed seed had a high moisture content, was still flowable and had less than 1% sprouted seed. The seed was primed for 7 days.

The germination results were obtained: as shown in Table 17.

TABLE 17
Thermogradient table test

| | Temp. (° C.) | | | | | | | | | | |
|---|---|---|---|---|---|---|---|---|---|---|---|
| | 16-18 | 18-20 | 20-22 | 22-24 | 24-26 | 26-28 | 28-30 | 30-32 | 32-34 | 34-36 | Ave |
| Treatment | 79 | 76 | 70 | 94 | 91 | 81 | 76 | 91 | 52 | 67 | 78 |
| primed not | | | | | | | | | | | |
| dried | | | | | | | | | | | |
| Treatment | 24 | 64 | 70 | 73 | 79 | 81 | 91 | 55 | 24 | 0 | 56 |
| untreated | | | | | | | | | | | |
| seed | | | | | | | | | | | |

The following compositions were made from the primed seed:
1. 100% primed seed
2. 70% primed seed/30% not primed seed
3. 100% not primed seed (=check)

These compositions were planted in 9 ft² plots, on a driving range of a golf course in San Francisco, Calif. (USA).

The visual observations as shown in Table 18 were made. From Table 18 it will be clear that the primed seed displays an improved emergence when compared with untreated seed. Photos of the results are attached.

TABLE 18
Visual inspection of field emergence made by the Assistant
Superintendent of the golf course:

| | | Plot #2 | | |
|---|---|---|---|---|
| | | 70% primed/ | Plot #3 | |
| Days after | Plot #1 | 30% non | 100% non | |
| planting | 100% primed | primed | primed | Remarks |
| 4 | Germinated, | Germinated, | Not | |
| | about 2-3 mm size | about 2-3 mm size | germinated | |
| | sprout | sprout | | |
| 5 | 30% | 20/25% | 0% | Looks like Plot #1 is |
| | | | | coming in a little |
| | | | | quicker |
| 6 | 45% | 45/50% | 10/15% | It seems that Plot #2 |
| | | | | has taken a jump over |
| | | | | Plot #1 |
| 7 | 60% | 65% | 35% | Both primed plots are |
| | | | | about equal in |
| | | | | coverage, even with the |
| | | | | control rye present in |
| | | | | Plot #2. |
| 9 | 75% | 75% | 60% | Plot #1 is still |
| | | | | germinating slowly |
| | | | | while Plots #2 & #3 are |
| | | | | advancing quite well |
| 10 | 75/80% | 75/80% | 65% | |
| 11 | 80% | 80% | 70% | |
| 12 | 85% | 85% | 75% | |
| 12 | 85/90% | 85/90% | 75/80% | Plot #3 is filling in |
| | | | | nicely and at a good |
| | | | | rate |
| 13 | 90% | 90% | 80% | |

### Example 12

A method according to the present invention was carried out as follows. Four samples of 50 g of Bent grass seed each, having a water content of 11.44% wt, were primed according to the method as previously described in Example 5. The solutions applied in the priming processes priming were as follows:
Solution 1: 0.2% KNO3 only
Solution 2: 0.2% KNO3+100 ppm GA3
Solution 3: 0.2% KNO3+100 ppm GA4+7
Solution 4: 0.2% KNO3+100 ppm GA3 and 100 ppm GA4+7

After 4 days of priming, evaluations of sprouted seeds were made and their results are shown in Table 19. It will be clear from Table 19 that the addition of additives (hormones) result in an increased number of sprouts.

TABLE 19
Sprouted seeds during priming as result of different solutions.

| priming solution | % of sprouted seeds |
|---|---|
| 1 | 49 |
| 2 | 52 |
| 3 | 56 |
| 4 | 60 |

1. A method for repairing, regrassing or establishing a green, which method comprises:
incorporating into a soil surface of the green, putting into holes in the soil surface, and/or applying at the soil surface, a flowable primed grass seed, which flowable primed grass seed displays physiological activity.

2. The method according to claim 1, wherein the grass seed is incorporated into the soil surface of the green to be prepared.

3. The method according to claim 1, wherein the primed grass seed is obtained by a process comprising:
subjecting a grass seed to a priming process after which the primed grass seed so obtained is immediately recovered for use to repair, regrass or establish a green or the primed grass seed so obtained is partially dehydrated before it is recovered for use to repair, regrass or establish a green.

4. The method according to claim 1, wherein the primed grass seed has a water content in the range of from 25 to 50% wt, based on total weight of seed.

5. The method according to claim 1, wherein the ratio of the water contents of the primed grass seed (A) and the grass seed to be primed (B) is greater than 1 (A/B).

6. The method according to claim 5, wherein A/B is in the range of from 1.6 to 10.0.

7. The method according to claim 6, wherein A/B is in the range of from 2 to 4.

8. The method according to claim 3, wherein less than 1% of the flowable primed grass seed displays radicles.

9. The method according to claim 3, wherein the primed grass seed is used to repair the green within 100 hours after the priming process has been completed.

10. The method according to claim 9, wherein the primed grass seed is used to repair the green within 24 hours after the priming process has been completed.

11. The method according to claim 1, wherein the primed grass seed has been coated.

12. The method according to claim 1, wherein an additive has been added to the primed grass seed.

13. The method according to claim 1, wherein the grass seed is selected from the group consisting of Poa, Lolium, Dactylis, Festuca, Deschampsia, Agrostis, Koeleria, Agrostis, Cynodon, Zoysia, Buchlo, Axonopus, Eremchloa, Paspalum and Stentaphrum.

14. The method according to claim 13, wherein the grass seed comprises Poa pratensis, Poa trivalis, Lolium perrenne, Agrostis capillaris tenuis, Agrostis stolonifera, or Festuca rubra or Festuca arundinacea.

15. The method according to claim 1, wherein the green is a golf green.

16. The method according to claim 15, wherein the green is a part of a teeing ground, fairway or putting green.

17. The method according to claim 1, wherein the primed grass seed comprises a coating which coating comprises an additive to improve the quality of the grass resulting therefrom.

18. The method according to claim 16, wherein, in addition, an additive to improve the quality of the grass is incorporated into the soil surfaces, put into holes in the soil surface, and/or applied at the soil surface.

19. The method according to claim 17, wherein the additive comprises a selective fertilizer.

20. The method according to claim 1, wherein, in addition, particles of a super absorbent polymer (a superslurper) are incorporated into the soil surface, put in holes into the surface, and/or applied at the soil surface.

21. A method of repairing, regressing, or establishing a green of the type involving use of grass seed, wherein the improvement comprises:
using, as the grass seed, flowable primed grass seed that displays physiological activity to repair, regrass or establish the green.

22. A flowable primed grass seed displaying physiological activity.
